(19) United States
(12) Patent Application Publication
(10) Pub. No.: US 2019/0175254 A1
(43) Pub. Date: Jun. 13, 2019

(54) ELECTROSURGICALLY REMOVING TISSUE WITH LOCALIZED RETURN

(71) Applicant: Covidien LP, Mansfield, MA (US)

(72) Inventor: JOHN R. TWOMEY, LONGMONT, CO (US)

(21) Appl. No.: 16/243,973

(22) Filed: Jan. 9, 2019

Related U.S. Application Data
(63) Continuation of application No. 14/798,104, filed on Jul. 13, 2015.
(60) Provisional application No. 62/042,523, filed on Aug. 27, 2014.

(51) Int. Cl. A61B 18/14

(52) U.S. Cl. CPC A61B 18/1442; A61B 2018/1226

(57) ABSTRACT

An electrosurgical system for removing tissue is provided. The electrosurgical system includes an electrosurgical device for treat tissue and a grasping device configured to grasp tissue. The electrosurgical device has one or more electrically conductive surfaces adapted to electrically couple to a first electrical potential of an electrosurgical energy source. The grasping device includes one or more electrically conductive surfaces adapted to electrically couple to a second electrical potential of the electrosurgical energy source wherein, upon activation of the electrosurgical energy source, electrosurgical energy is transmitted between electrical potentials and through tissue disposed therebetween.

### CROSS-REFERENCE TO RELATED APPLICATIONS

The present application is a continuation application of U.S. patent application Ser. No. 14/798,104, filed on Jul. 13, 2015, which claims the benefit of and priority to U.S. Provisional Patent Application No. 62/042,523, filed on Aug. 27, 2014, the entire contents of each of which are hereby incorporated herein by reference.

### TECHNICAL FIELD

The present disclosure relates to surgical devices, systems, and methods for performing tonsillectomies present disclosure relates to surgical devices, systems, and methods for performing tonsillectomies and adenoidectomies, and, more particularly, to surgical devices systems, and methods with localized return features utilized for tonsillectomies and adenoidectomies.

### BACKGROUND

The tonsils and adenoids are part of the lymphatic system and are generally located in the back of the throat. These parts of the lymphatic system are generally used for sampling bacteria and viruses entering the body and activating the immune system when warranted to produce antibodies to fight oncoming infections. More particularly, the tonsils and adenoids break down the bacteria or virus and send pieces of the bacteria or virus to the immune system to produce antibodies for fighting off infections.

Inflammation of the tonsils and adenoids (e.g., tonsillitis) impedes the ability of the tonsils and adenoids to destroy the bacteria resulting in a bacterial infection. In many instances, the bacteria remain even after treatment and serve as a reservoir for repeated infections (e.g., tonsillitis or ear infections).

A tonsillectomy and/or adenoidectomy may be indicated when infections persist and antibiotic treatments fail. Persistent infection typically leads to enlarged tonsil tissue which may need to be removed since in many cases the enlarged tissue causes airway obstruction leading to various sleep disorders such as snoring or, in some cases, sleep apnea. Some individuals are also born with larger tonsils that are more prone to cause obstruction. An adenoidectomy may also be required to remove adenoid tissue when ear pain persists, or when nose breathing or function of the eustachian tube (a.k.a. auditory or pharyngotympanic tube) is impaired. Many surgeons prefer to perform these two procedures at the same time.

The type of surgical device and/or method used for tonsillectomies and adenoidectomies usually depends on the type and amount of tissue to be removed and/or a surgeon's preference. Various methods for performing a tonsillectomy and/or adenoidectomy employ an array of surgical instrumentation and, in most cases, a variety of energy modalities to accomplish the underlying purpose of removing the infected tissue. Technologies include, cold dissection, monopolar and bipolar diathermy dissection, dissection using bipolar scissors, laser tonsillectomy, cryosurgery, ultrasonic removal, microdebrider, Coblation® and so-called thermal welding. A scalpel or other sharp instrument such as a curette or punch device may also be used to remove tissue but using these types of instruments typically results in heavy bleeding which needs to be stemmed with electrocautery.

Other electrosurgical devices may also be employed such as suction-tipped devices, blades, or needle tip devices, e.g., various Bovie devices. A Bovie device typically has a hollow center to suction blood, secretions, and smoke from the surgical field, and a rim of metal for cutting and coagulation. A separate aspirator is used when blade and needle tip Bovies are used. Although the use of Bovies reduces blood loss intraoperatively in comparison to various known cold techniques, its use may be associated with an increase in postoperative pain due to thermal spread from the heat created during use (e.g., above 3000° C.). Despite a large thermal injury profile, use of the Bovie surgical instrument remains very popular for tonsil removal.

Some commercial attempts have been made to limit or minimize thermal injury. These include: the HARMONIC SYNERGY® Blades (Ethicon Endo-Surgery, Cincinnati, Ohio) (ultrasonic energy), lasers (e.g., KTP, Nd: YAG, or CO2 lasers), and Coblation® devices (Arthrocare, Austin, Tex.) (bipolar radiofrequency ablation). However, the decrease in thermal injury provided by these devices remains questionable and is offset by a reduced control of bleeding and surrounding tissue trauma, longer operative times, introduction of fluids (e.g., saline) and/or less precise cutting. Some of the instruments also obscure the surgical field and are difficult to maneuver due to their large size.

### SUMMARY

Accordingly, new devices for resecting tonsil and adenoid tissue would be useful. In particular, devices that precisely cut tonsil and adenoid tissue while effectively controlling bleeding and surrounding tissue trauma would be desirable. Devices that provide easier access to the tonsils and adenoids and manipulation of those tissues would also be desirable. Moreover, it would also be useful to have devices particularly suited for treating and/or removing the underlying tissue or tissue within the tonsil bed which, in many instances, can lead to serious concerns. As mentioned above, the tonsil bed, if not properly treated or removed, acts as a reservoir for bacteria leading to repeated infections.

In one aspect, the present disclosure relates to an electrosurgical system for treating and/or removing tissue. The electrosurgical system includes an electrosurgical device for treating tissue and a grasping device configured to grasp tissue.

The electrosurgical device has one or more electrically conductive surfaces adapted to electrically couple to a first electrical potential of an electrosurgical energy source. The electrosurgical energy source may be powered by a battery. The electrosurgical device may be a monopolar pencil.

The grasping device includes one or more electrically conductive surfaces adapted to electrically couple to a second electrical potential of the electrosurgical energy source wherein, upon activation of the electrosurgical energy source, electrosurgical energy is transmitted between electrical potentials and through tissue disposed therebetween. In embodiments, the grasping device is adapted to couple to a suction source and a distal end of the grasping device includes the one or more electrically conductive surfaces that engage tissue under suction. In some embodiments, the grasping device includes a pair of jaw members selectively movable between an open condition and a closed condition for grasping tissue therebetween. One or both of the pair of jaw members can include the one or more electrically conductive surfaces. In embodiments, the grasping device is a clamp. In some embodiments, the grasping device has an Allis clamp configuration.

According to another aspect, an electrosurgical system for removing tissue includes an electrosurgical energy source, an electrosurgical device, and a grasping device.

The electrosurgical device has one or more electrically conductive surfaces electrically coupled to a first potential of the electrosurgical energy source. The one or more electrically conductive surfaces of the electrosurgical device is configured to transmit electrosurgical energy of a first potential therefrom.

The grasping device includes first and second opposing jaw members configured to grasp tissue therebetween. The grasping device includes one or more electrically conductive surfaces disposed on one or both of the jaw members. The grasping device is configured to connect to a second potential of the electrosurgical energy source wherein, upon activation of the electrosurgical energy source, electrosurgical energy travels from the one or more electrically conductive surfaces of the electrosurgical device, through tissue grasped by the grasping device, and to the one or more electrically conductive surfaces of the grasping device to complete a circuit.

In some embodiments, the electrosurgical energy source is a generator. The generator may be powered by a battery. In certain embodiments, the generator is powered by alternating current.

According to yet another aspect, the present disclosure relates to a method for removing tissue. The method involves grasping tissue with a grasping device; conducting electrosurgical energy through the tissue between an electrosurgical device electrically coupled to a first potential of an electrosurgical energy source, and the grasping device electrically coupled to a second potential of the electrosurgical energy source; and manipulating the electrosurgical device to cut the grasped tissue from remaining tissue and removing grasped tissue. The method may involve conducting energy through a return electrode coupled to a patient. The method may include limiting electrical conductance through the return electrode with load resistance coupled to the return electrode.

In some embodiments, the electrosurgical device can include an electrosurgical pencil having a first electrically conductive surface and the grasping device can includes a clamp having a second electrically conductive surface, wherein conducting electrosurgical energy can include conducting the electrosurgical energy between the first and second electrically conductive surfaces.

In certain embodiments, the electrosurgical device includes a pair of jaw members having a first electrically conductive surface and the grasping device includes a clamp having a second electrically conductive surface, wherein conducting electrosurgical energy can include conducting the electrosurgical energy between the first and second electrically conductive surfaces.

Other aspects, features, and advantages will be apparent from the description, the drawings, and the claims that follow.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiment(s) given below, serve to explain the principles of the disclosure, wherein:

FIG. 1 is a perspective view of an electrosurgical system according to the principles of the present disclosure;

FIGS. 2A-2C are progressive schematic views illustrating removal of tissue with the electrosurgical system of FIG. 1; and

FIGS. 3A-3C are progressive schematic views illustrating removal of tissue with another embodiment of an electrosurgical system.

### DETAILED DESCRIPTION

Particular embodiments of the present disclosure are described hereinbelow with reference to the accompanying drawings in which like reference numerals designate identical or corresponding elements in each of the several views. As used herein the term “distal” refers to that portion of the system, apparatus and/or device, or component thereof, that are farther from the user, while the term “proximal” refers to that portion of the system, apparatus and/or device, or component thereof, that are closer to the user. In the following description, well-known functions or constructions are not described in detail to avoid obscuring the present disclosure in unnecessary detail.

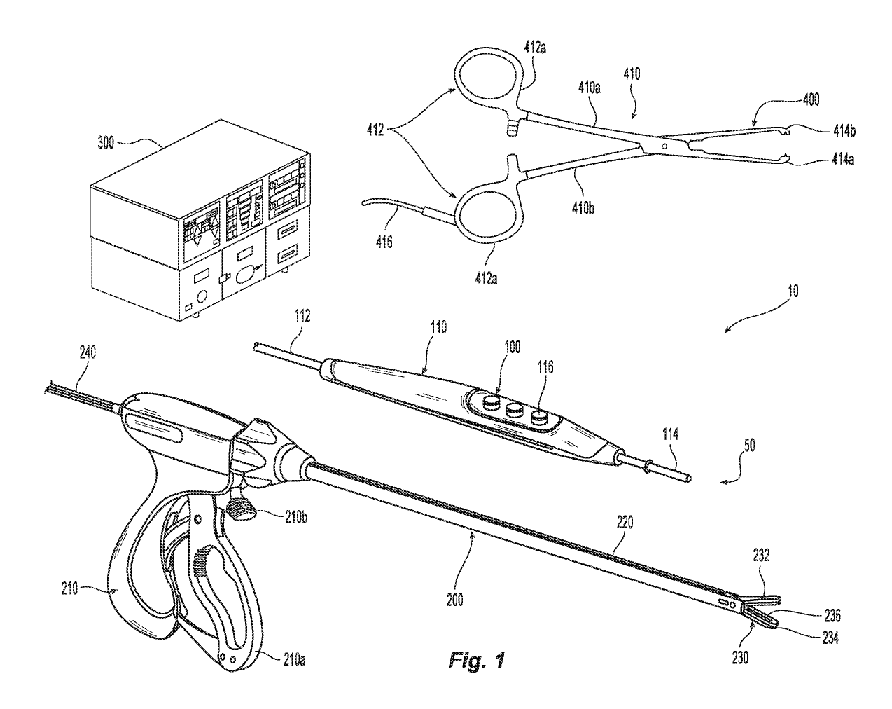

Turning now to FIG. 1, an electrosurgical system, in accordance with the present disclosure, generally referred to as 10, includes an electrosurgical device such as an electrosurgical pencil 100 and/or an electrosurgical forceps 200 that electrically couple to an electrosurgical energy source 300 (e.g., a generator), and a grasping device 400 that also electrically couples to electrosurgical energy source 300.

Electrosurgical devices such as electrosurgical pencil 100 generally include a body 110 having a supply line 112 extending from a proximal end thereof and one or more active electrodes and/or electrically conductive surface(s) 114 (e.g., electrosurgical cutting probe, ablation electrode(s), etc.) extending from a distal end thereof that are configured to electrical communicate with supply line 112. The supply line 112 connects to an active terminal of electrosurgical energy source 300 at a proximal end of the supply line 112. One or more actuators 116 (e.g., buttons, switches, knobs, slides, etc.) are coupled to body 110 to control electrical communication between supply line 112 and electrically conductive surface(s) 114 for selectively activating electrically conductive surface(s) 114 to treat tissue (e.g., cut, coagulate, ablate, etc.)

Electrosurgical devices such as electrosurgical forceps 200 generally include a handle 210 having a distally extending shaft 220 that supports an end effector 230 on a distal end of shaft 220. End effector 230 includes a pair of jaw members having an upper jaw member 232 and a lower jaw member 234 that are movable/pivotable between open and closed conditions upon an actuation of a jaw trigger 210a for grasping tissue therebetween. One or both of the pair of jaw members 232, 234 include a sealing surface 236 that electrically communicates with a supply line 240 extending proximally from a proximal end of handle 210. An activation trigger 210b is actuatable to provide electrical communication between sealing surface 236 and supply line 240 for selectively activating electrically conductive surface 236 to treat tissue (e.g., cut, coagulate, ablate, etc.).

As can be appreciated, any of these electrosurgical devices can be monopolar and/or bipolar.

Electrosurgical energy sources such as electrosurgical energy source 300 can perform monopolar and/or bipolar electrosurgical procedures, including, for example, cutting, coagulation, ablation, and vessel sealing procedures. The electrosurgical energy source 300 may include a plurality of outputs for interfacing with various electrosurgical instruments such as electrosurgical pencil 100, electrosurgical forceps 200, grasping device 400 and/or any other suitable energy modality. Electrosurgical energy source 300 may be configured to interface with any suitable electrosurgical instrument (e.g., a monopolar instrument, return electrode, bipolar electrosurgical forceps, footswitch, etc.). Further, the electrosurgical energy source 300 can include any suitable electronic circuitry. For example, electrosurgical energy source 300 can include electronic circuitry configured to generate radio frequency energy specifically suited for various electrosurgical modes (e.g., cut, blend, coagulate, division with hemostasis, fulgurate, spray, etc.) and procedures (e.g., monopolar, bipolar, vessel sealing). In embodiments, electrosurgical energy source 300 may be embedded, integrated or otherwise coupled to the electrosurgical and/or grasping devices providing for an all-in-one electrosurgical apparatus/system. In embodiments, electrosurgical energy source 300 may be powered by a battery or powered by alternating current. Electrosurgical energy source 300 may include one or more converting devices for converting from DC to AC or vice a versa.

Grasping device 400 includes a body 410 including a first arm 410a and a second arm 410b that are pivotally coupled together to form a handle portion 412 at a proximal end thereof and a pair of jaw members 414a, 414b at a distal end thereof. Handle portion 412 is operable to selectively move/pivot the pair of jaw members 414a, 414b between open and closed conditions for grasping tissue, such as throat tissue, therebetween. At least a portion of body 410 can include electrically conductive material (e.g., metallic material or the like) which can be an electrically conductive surface. In some embodiments, only portions of body 410 include electrically conductive materials and/or surfaces, such as, for example, one or both of the pair of jaw members 414a, 414b. In certain embodiments, the entire body 410 is electrically conductive. Body 410, or portions thereof, can include insulative material which may be provided as a layer or coating. For example, handle portion 412 can include a dielectric cover 412a positioned on electrically conductive surfaces of handle portion 412.

A return line 416 extends proximally from body 410 and is disposed in electrical communication with the electrically conductive material/surface(s) of body 410. Return line 416 is configured to couple the electrically conductive material/surfaces of body 410 to electrosurgical energy source 300 (e.g., via a return terminal of electrosurgical energy source 300) for electrically communicating with electrosurgical energy source 300. In this manner, grasping device 400 can function as an electrical return for electrosurgical energy (e.g., electric current) conducted through/transmitted from one or more of the electrosurgical devices 100, 200 of the present disclosure as will be described in greater detail below. Embodiments of grasping device 400 can include configurations of Allis clamps, Babcock clamps, hemostats, tweezers, forceps, and other suitable graspers.

Electrosurgical devices 100, 200 can be configured to conduct/transmit any suitable electric current (e.g., AC and/or DC) at any suitable frequency. In embodiments, one or more of the presently described devices 100, 200 can be configured to provide radio frequency (RF) energy. For a detailed discussion of the construction and operation of example electrosurgical devices, electrosurgical energy sources, and/or grasping devices, reference may be made to U.S. Patent Application Publication No. 2013/0267947, U.S. Patent Application Publication No. 2013/0255063, U.S. Pat. No. 7,156,844, U.S. Pat. No. 5,766,167, and/or U.S. Pat. No. 5,026,370, each of which is incorporated herein by reference.

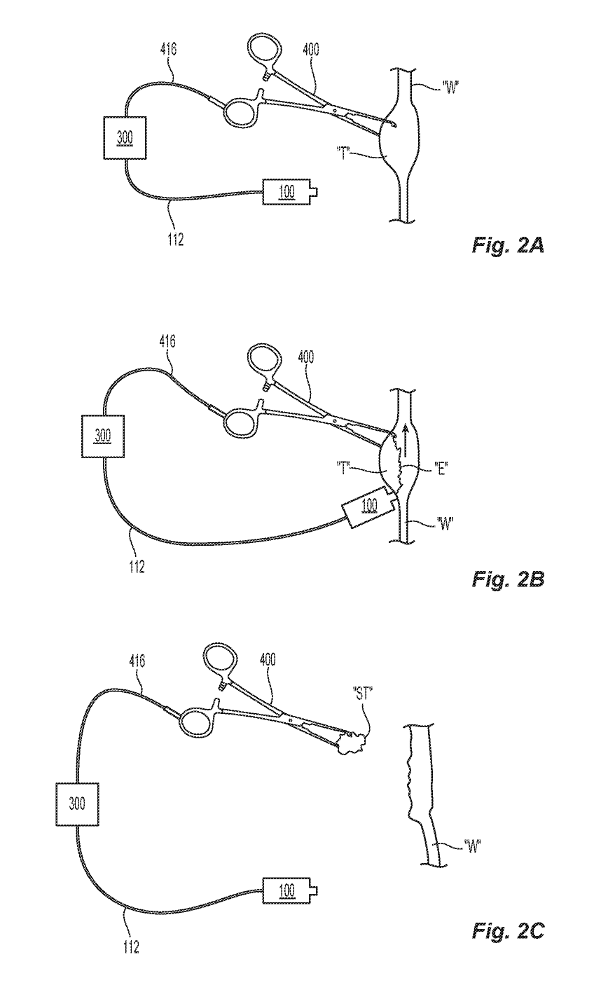

In operation, as seen in FIGS. 2A-2C, grasping device 400 is operable to grasp tissue “T” disposed on a wall “W” so that electrosurgical energy “E” (e.g., current) can be conducted through tissue “T” while electrosurgical device (e.g., electrosurgical pencil 100) is disposed adjacent to the tissue “T.” In the illustrated embodiment, tissue “T” is throat tissue such as tonsil or adenoid tissue; however, the presently described devices, systems, and methods can be applied to any suitable tissue.

To remove the tissue “T” from the wall “W,” activation of electrosurgical pencil 100 draws the electrosurgical energy “E” from electrosurgical energy source 300 through supply line 112 and directs the electrosurgical energy “E” into the tissue “T.” Grasping device 400 functions as a return electrode for the electrosurgical energy “E,” returning the electrosurgical energy “E” to electrosurgical energy source 300 through return line 416. In this regard, the electrosurgical energy “E” applied via electrosurgical pencil 100 across the tissue “T” severs the tissue “T” from the wall “W.” Thus, with severed tissue “ST” grasped by the grasping device 400, the severed tissue “ST” can be removed and the process can be repeated as necessary to remove any undesirable tissue “T” using, for example, intracapsular (e.g., subtotal tonsillectomy) and/or extracapsular (e.g., total tonsillectomy) techniques.

After tissue “T” is removed, a clinician may need to coagulate and/or cauterize uncontrolled bleeding (“bleeders”). As such, a separate return electrode (e.g., a return pad, not shown) may be positioned on the patient to act as a return pathway for electrosurgical energy. The return electrode may include a load resistor, e.g., 50 ohm load resistor (not shown), that prevents the return electrode from serving as the return pathway until after tissue “T” is removed. In this regard, thermal damage to underlying tissue is minimized while enabling a clinician to coagulate and/or cauterize bleeders after removal of tissue “T.”

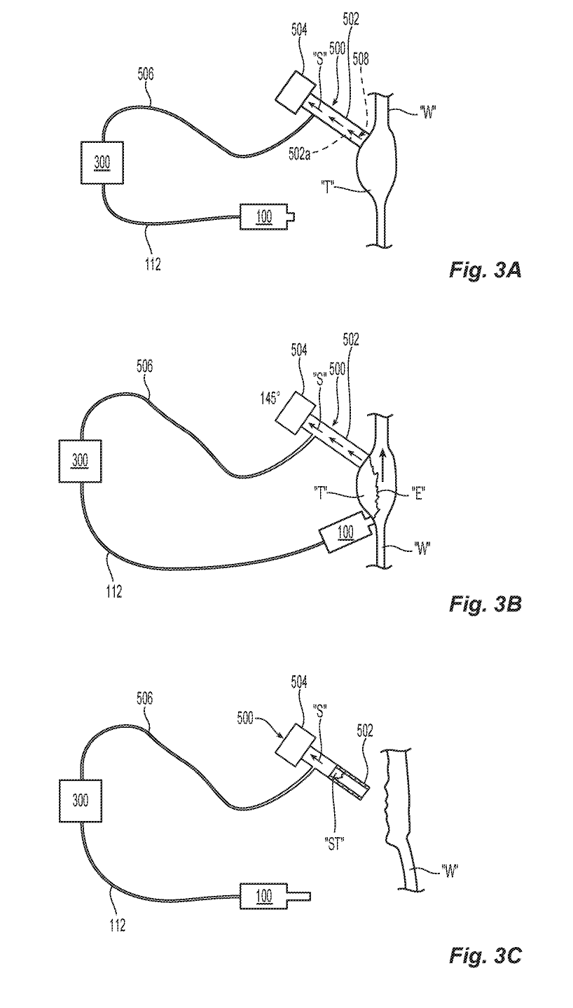

Turning now to FIGS. 3A-3C, one embodiment of a grasping device, generally referred to as 500, includes a grasping member 502 in fluid communication with a vacuum/suction source 502 and electrically coupled to a return line 506 in electrical communication with electrosurgical energy source 300. Suction source 502 is configured to apply suction “S” through a lumen 508 defined by inner surfaces 502a of grasping member 502. Grasping member 502 can be wholly, or partially, formed of electrically conductive material.

In operation, grasping device 500 is operable to grasp tissue “T” (e.g., via suction “S” from suction source 504) disposed on a wall “W” so that electrosurgical energy “E” can be conducted through tissue “T” while electrosurgical device (e.g., electrosurgical pencil 100) is disposed adjacent to the tissue “T” similar to that described above. In particular, to remove the tissue “T” from the wall “W,” activation of electrosurgical pencil 100 draws the electrosurgical energy “E” from electrosurgical energy source 300 through supply line 112 and directs the electrosurgical energy “E” into the tissue “T.” Grasping device 500 functions as a return electrode for the electrosurgical energy “E,” returning the electrosurgical energy “E” to electrosurgical energy source 300 through return line 506. In this regard, the electrosurgical energy “E” applied via electrosurgical pencil 100 across the tissue “T” severs the tissue “T” from the wall “W” so that suction “S” draws severed tissue “ST” through lumen 508 of grasping member 502 for removal. This process can be repeated as necessary to remove any undesirable tissue “T” using, for example, intracapsular and/or extracapsular techniques.

The various embodiments disclosed herein may also be configured to work with robotic surgical systems and what is commonly referred to as “Telesurgery”. Such systems employ various robotic elements to assist the surgeon in the operating theatre and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include, remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prep the patient for surgery and configure the robotic surgical system with one or more of the instruments disclosed herein while another surgeon (or group of surgeons) remotely control the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

Persons skilled in the art will understand that the structures and methods specifically described herein and shown in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure. Additionally, the elements and features shown or described in connection with certain embodiments may be combined with the elements and features of certain other embodiments without departing from the scope of the present disclosure, and that such modifications and variations are also included within the scope of the present disclosure. Accordingly, the subject matter of the present disclosure is not limited by what has been particularly shown and described.

1-17. (canceled)

18. An electrosurgical system for removing tissue, comprising:
an electrosurgical energy source including an active monopolar terminal and a return monopolar terminal;
an electrosurgical instrument including a supply line configured to connect to the active monopolar terminal of the electrosurgical energy source; and
a grasping device including a return line configured to connect to the return monopolar terminal of the electrosurgical energy source,
wherein the electrosurgical energy source, the electrosurgical instrument, and the grasping device are configured to create a monopolar electrosurgical circuit to enable delivery of monopolar energy from the electrosurgical energy source to the electrosurgical instrument to tissue and return of monopolar energy from tissue to the grasping device to the electrosurgical energy source.

19. The electrosurgical system according to claim 18, wherein the electrosurgical instrument is a monopolar electrosurgical pencil.

20. The electrosurgical system according to claim 18, wherein the grasping device includes first and second arms coupled together about a pivot to define first and second handles proximally of the pivot and first and second jaw members distally of the pivot, wherein the handles are pivotable relative to one another to thereby pivot the first and second jaw members relative to one another between an open position and a closed position.

21. The electrosurgical system according to claim 20, where each of the first and second jaw members is disposed in electrical communication with the return line.

22. The electrosurgical system according to claim 18, wherein the electrosurgical energy source is an electrosurgical generator.

23. The electrosurgical system according to claim 22, wherein the electrosurgical generator is configured to supply RF energy via the active monopolar terminal and receive RF energy via the return monopolar terminal.

24. The electrosurgical system according to claim 22, wherein the electrosurgical generator further includes a bipolar terminal.

25. A method of setting up an electrosurgical system, the method comprising:
connecting an electrosurgical instrument to an active monopolar terminal of an electrosurgical energy source; and
connecting a grasping device to a return monopolar terminal of the electrosurgical energy source.

26. The method according to claim 25 wherein connecting the electrosurgical instrument includes connecting a supply line of the electrosurgical instrument to the active monopolar terminal.

27. The method according to claim 25, wherein coting the grasping device includes connecting a return line of the grasping device to the return monopolar terminal.

28. The method according to claim 25, wherein the electrosurgical instrument is a monopolar electrosurgical pencil.

29. The method according to claim 25, wherein the grasping device includes first and second arms coupled together about a pivot to define first and second handles proximally of the pivot and first and second jaw members distally of the pivot, wherein the handles are pivotable relative to one another to thereby pivot the jaw members relative to one another between an open position and a closed position.

30. The method according to claim 29, wherein each of the first and second jaw members is configured to electrically connect to the return monopolar terminal of the electrosurgical energy source.

31. The method according to claim 25, wherein the electrosurgical energy source is an electrosurgical generator.

32. The method according to claim 31, wherein the electrosurgical energy source is configured to supply RF energy via the active monopolar terminal and receive RF energy via the return monopolar terminal.

33. The method according to claim 31, wherein, the electrosurgical generator further includes a bipolar terminal.

34. A method of surgery, the method comprising:
grasping tissue with a grasping device including a return line connected to a return monopolar terminal of an electrosurgical energy source;
contacting tissue with an electrosurgical instrument including a supply line connected to an active monopolar terminal of the electrosurgical energy source; and
activating the electrosurgical instrument to create a monopolar electrosurgical circuit wherein monopolar energy is delivered from the electrosurgical energy source to the electrosurgical instrument to tissue and returned from tissue to the grasping device to the electrosurgical energy source.

35. The method according to claim 34, further comprising electrosurgically cutting the grasped tissue with the monopolar energy to sever the grasped tissue from other tissue.

36. The method according to claim 35, further comprising removing the severed, grasped tissue using the grasping device.

37. The method according to claim 35, wherein electrosurgically cutting the grasped tissue includes moving an electrode of the electrosurgical instrument across tissue.
